(19) United States
(12) Patent Application Publication
(10) Pub. No.: US 2021/0219514 A1
(43) Pub. Date: Jul. 22, 2021

(54) FILANTHERLESS AFRICAN VIOLET AND METHODS OF BREEDING THEREOF

(71) Applicant: HERMANN HOLTKAMP GREENHOUSES, INC., Nashville, TN (US)

(72) Inventors: Reinhold HOLTKAMP, Jr., Brentwood, TN (US); Martin HOLTKAMP, Isselburg-Vehlingen (DE)

(73) Assignee: HERMANN HOLTKAMP GREENHOUSES, INC., Nashville, TN (US)

(21) Appl. No.: 17/259,851

(22) PCT Filed: Jul. 12, 2019
(86) PCT No.: PCT/US2019/041702

§ 371 Date: Jan. 12, 2021

Related U.S. Application Data
(60) Provisional application No. 62/697,792, filed on Jul. 13, 2018.

(51) Int. Cl. A01H 6/44; A01H 5/02

(52) U.S. Cl. CPC A01H 6/444; A01H 5/02

(57) ABSTRACT

The disclosure provides a Saintpaulia plant comprising at least one flower that is absent of a stamen in the flower and a method of producing the Saintpaulia plant.

### CROSS-REFERENCE TO RELATED APPLICATION

This application claims the benefit of U.S. Provisional Application No. 62/697,792 filed Jul. 13, 2018, the content of which is incorporated herein by reference in its entirety.

### TECHNICAL FIELD

The present invention relates to new, distinct and stable cultivars of Saintpaulia (i.e., African violets) that has a mature flower without any filaments or anthers visible on the flowers—“filantherless.” The flower does not show the male reproductive organs comprised of the filament and anther also known as the Stamen. The present invention also relates a method of producing the filantherless Saintpaulia.

### BACKGROUND

As one of the most popular and widely enjoyed house plants, African violets (Saintpaulia) are grown for sale as domestic house plants. Many types of African violets have been developed through controlled breeding programs leading to desirable and stable characteristics. Among the characteristics commercial breeders strive to develop in new cultivars through traditional breeding are attractive flower colors, profuse flowering and a long flowering period, among others.

Thus far, traditional breeding techniques have failed to produce African violet cultivars that are stable in producing flowers, resistant to insects of pathogens during their growth and delivery to customers. One of the most challenging insects in production of the African violet is western flower thrip (Frankiniella occidentalis). The insects are about 1 mm in size and can enter the anther for protection. To allow for sufficient space, the insects remove the pollen within the anther. The removal often spills the pollen over the flower petals, which makes the flower unsightly. Also plants with the presence of Frankiniella occidentalis are labeled unsellable. Furthermore, Frankiniella occidentalis is also a piercing insect that can pierce the flower for nutrients. Such piercing causes the flower to wilt prematurely. The wilted flowers during an infestation need to be removed before the mature plant can be prepared for the market and shipping.

The anther can also create a challenge when boxed for shipping. The anther tissue is a perfect breeding ground for the fungal pathogen called Botrytis. This fungus grows quickly in conditions during delivery or shipping, e.g., high humidity and lack of ventilation within the shipping box. Even after a short period of time in the boxes, the anther may show fuzzy furballs of the botrytis fungus, which also makes the plant unsellable. Thus, the plants are sprayed preemptively with a fungicide to control the fungal growth before the plants are packaged for shipping. This preemptive approach, however, does not work on the plants all the time.

Attempts have been made to increase the number of flowers, colors, flower shapes and sizes, and even the presence of multiple flower stems in order to improve the appeal to the consumers. But the past breeding attempts have failed to prevent parasites that pose challenges to the African violets. Therefore, there exist a need to develop a new strain of African violets that are more resistant to flower parasites, e.g., Frankliniella occidentalis and Botrytis.

### SUMMARY OF INVENTION

In one aspect, the disclosure provides a Saintpaulia plant comprising at least one flower that is absent of a stamen in the flower. In one embodiment, the flower lacks a filament and an anther during the first flash of the flower. In another embodiment, the filament and the anther develop during the second flash of the flower. In another embodiment, the plant is produced from seeds having ATCC deposit Accession No. PTA ______. In another embodiment, the Saintpaulia plant develops a stamen with a filament and an anther during the second or later flash of flowers. In a different embodiment, the Saintpaulia plant is less likely to be infected with a pathogen than a control. The pathogen is one or more of botrytis and Frankiniella occidentalis.

In another aspect, the disclosure provides a method of producing a Saintpaulia plant that comprises at least one mature flower that is absent of a stamen in the flower, and the method comprises crossing a first Saintpaulia plant that has partial filament and antherless characteristics with a second Saintpaulia plant having a second desirable trait. In one embodiment, the second Saintpaulia plant has a fully developed stamen. In another embodiment, the second Saintpaulia plant has a filament and an anther. In another embodiment, the second desirable trait is selected from the group consisting of flower color, leaf color, disease resistance, leaf size and growth habit. In another embodiment, the method further comprises producing a seed from the Saintpaulia plant that comprises at least one mature flower that is absent of a stamen in the flower, wherein the seed is capable of growing to a filantherless Saintpaulia plant. In one embodiment, the method comprises selecting a progeny from the crossing, wherein the progeny produces more filantherless flowers than its parents. In one embodiment, the second Saintpaulia plant has a fully developed stamen. In another embodiment, the second Saintpaulia plant has a filament and an anther. In one embodiment, wherein the second desirable trait comprises one or more of flower color, leaf color, disease resistance, leaf size and growth habit. In another embodiment, the method further comprises producing a seed from the Saintpaulia plant that comprises at least one mature flower that is absent of a stamen in the flower, wherein the seed is capable of growing to a filantherless Saintpaulia plant. In one embodiment, the method further comprises selecting a progeny from the crossing, wherein the progeny produces more filantherless flowers than its parents.

The foregoing general description and following brief description of the drawings and the detailed description are exemplary and explanatory and are intended to provide further explanation of the invention as claimed. Other objects, advantages and novel features will be readily apparent to those skilled in the art from the following detailed description of the invention.

### BRIEF DESCRIPTION OF THE DRAWINGS

FIG. 1 shows a drawing of a typical Saintpaulia plant.

FIG. 2 shows a diagram of showing various parts of an African violet flower.

FIG. 3 shows the cultivar ‘5050 bl w KS 15’ that has the filantherless trait and was the preferred breeding stock for four new cultivars exhibiting the filantherless trait. The flower in the figure is without the stamen or the filament and anther.

FIG. 4A shows a photograph of the flower from ‘5050 Flied KS 14’ without the stamen or the lack of development of the filament and anther. FIG. 4B shows the filantherless characteristic. Arrows point to small underdeveloped filament with the first flash of flowers.

FIG. 5 shows the genealogy of the filantherless programs and the cultivars 5050 bl w KS15, 5043/7 and 5056.

FIG. 6 shows a flower infested with the western flower thrip (Frankliniella occidentalis). In the picture, the western flower thrip has overtaken the anthers and spilled all the pollen over the flower petals. The thirp is also a sucking insect which can pierce the flower petals and make additional permanent damage. Arrows point to the western flower thrips on the flower. The damage done by the western flower thrips is circled out.

FIGS. 7A and 7B show an infestation of the Botrytis cinereal fungus. The exterior tissue of the anther is a favorable breeding ground for the fungus, which affects the anthers under the right conditions. Once the flower is infected by the fungus, the flower will die shortly after the symptoms show on the anther.

### DETAILED DESCRIPTION

After reading this description, it will become apparent to one skilled in the art how to implement the disclosure in various alternative embodiments and alternative applications. However, not all embodiments of the present disclosure are described herein. It will be understood that the embodiments presented here are presented by way of an example only, and not limitation. As such, this detailed description of various alternative embodiments should not be construed to limit the scope or breadth of the present disclosure as set forth below.

Before the present disclosure is disclosed and described, it is to be understood that the aspects described below are not limited to specific cultivars, methods of preparing or breeding such cultivars, or uses thereof, as such may, of course, vary. It is also to be understood that the terminology used herein is for the purpose of describing particular aspects only and is not intended to be limiting.

The terminology used herein is for the purpose of describing particular embodiments only and is not intended to be limiting of the disclosure. As used herein, the singular forms “a,” “an” and “the” are intended to include the plural forms as well, unless the context clearly indicates otherwise.

All numerical designations, e.g., pH, temperature, time, concentration, amounts and molecular weight, including ranges, are approximations which are varied (+) or (−) by 10%, 1% or 0.1%, as appropriate. It is to be understood, although not always explicitly stated, that all numerical designations may be preceded by the term “about.” It is also to be understood, although not always explicitly stated, that the reagents described herein are merely exemplary and that equivalents of such are known in the art.

The term “about,” when used before a numerical designation, e.g., temperature, time, amount, concentration and such others, including a range, indicates approximations which may vary by (+) or (−) 10%, 5%, 1% or any subrange or subvalue there between. Preferably, the term “about” when used with regard to a dose amount means that the dose may vary by +/−10%.

The term “comprising” or “comprises” is intended to mean that the compositions and methods include the recited elements but do not exclude others. “Consisting essentially of,” when used to define compositions and methods, shall mean excluding other elements of any essential significance to the combination. For example, a composition consisting essentially of the elements as defined herein would not exclude other elements that do not materially affect the basic and novel characteristic(s) of the claimed disclosure. “Consisting of” shall mean excluding more than trace amounts of other ingredients and substantial method steps recited. Embodiments defined by each of these transition terms are within the scope of this disclosure.

The term “filantherless,” as used here, refers to a flower that is missing the filament and anther that are normally found in a mature flower. In one embodiment, the flower is filantherless at least at its first flash. In another embodiment, the flower is filantherless at least at its first and second flashes. In another embodiment, the flower is filantherless at least at its first, second and third flashes. In another embodiment, the flower is filantherless at its first flash but retains its stamen, including a filament and/or anther at its second flash. In one embodiment, the flower is filantherless after one or more generations of asexual reproduction.

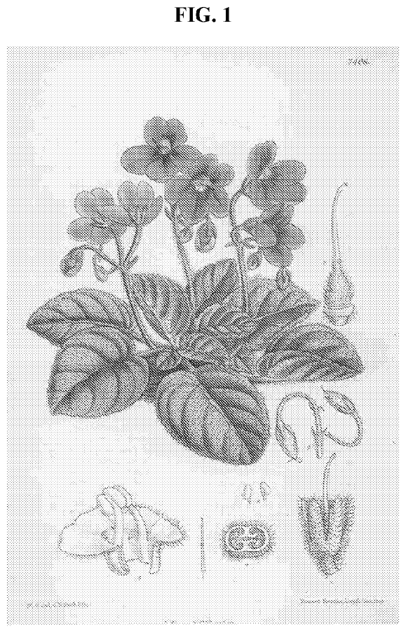

The instant invention provides a new variety of Saintpaulia plant with a stable trait—filantherless flower. The remaining part of the plant still maintains the typical Saintpaulia flower characteristics of both sexes (FIG. 1).

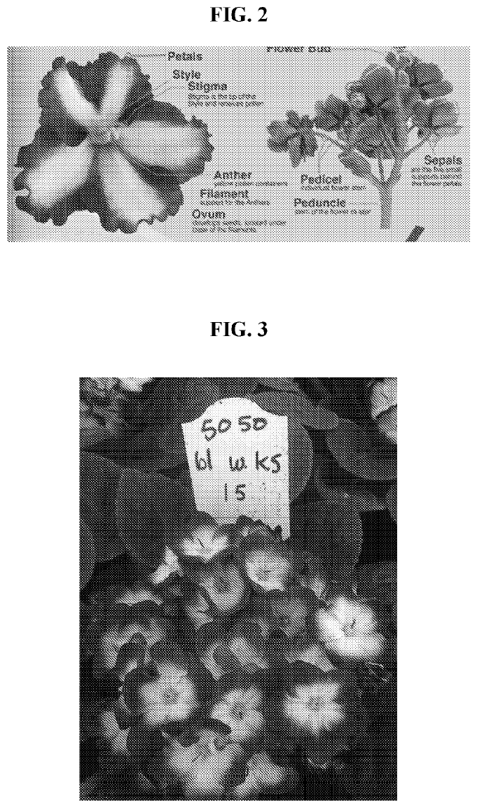

A typical Saintpaulia plant includes a style, a stigma, an ovum and the male stamen, which is composed of a filament and an anther (FIG. 2). The filaments and the anthers are missing in the claimed embodiments. The lack of the stamen without the filaments and anthers has great benefit because it limits the excessive use of chemicals in getting the Saintpaulia plant ready for the market. Since the 1980s, the emergence of the Western Flower Thrip or Frankliniella occidentalis set the stage for a long-time battle against this tiny invasive insect. Many of the known chemicals at the time were not effective in treating the problem. In a commercial greenhouse, the first-generation flower stem is essential in the production of the Saintpaulia plant for the market. The absence of stamen and its filament and anther has no or little adverse visual effect as compared to a typical Saintpaulia plant in terms of the consumer preference. However, the presence of the anthers presents a great challenge for the commercial grower as the anthers can attract insects, pathogens and/or fungus and thus requires additional cost or efforts to control insects, pathogens and fungus.

Thus, a filantherless trait is a greatly beneficial flowering characteristic. Limited close tests have shown that lack of anthers is not noticed by the observer. The primary focus remains on the flower shape and flower color, and only the most astute observer would notice that the filament and anthers are missing. The presence of anthers with the troubling Western Flower Thrip (Frankliniella occidentalis) or the presence of the pathogen Botrytis is more troubling to keep the plants sellable. Past research has not focused on eliminating the filament or the anther with the benefit of using less chemicals during the production of the Saintpaulia cultivars yet still preserving the appeal to the consumer without them noticing.

There is a need for new strategies for producing the Saintpaulia plants without the development of the stamen featuring the filament and anthers so the plant does not need to have excessive chemicals applied for insect or pathogen control. Furthermore, the strategy is to produce the Saintpaulia plants to full flower without the need to protect the plant from Botrytis and for shipping to the markets. With this new filantherless trait, the flowers have an increased numbers of flower stems, a larger numbers of flowers and a more continuous flowering habit, each of which constitutes a great improvement to the development of the African violet. Reduction of waste in the production and at the retailers, where apparent thrip damage or pathogens often makes the flowers unsellable, allows for an improved yield and reduces the cost of production and selling.

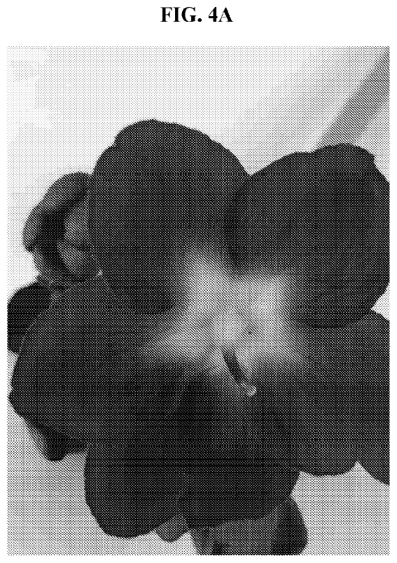

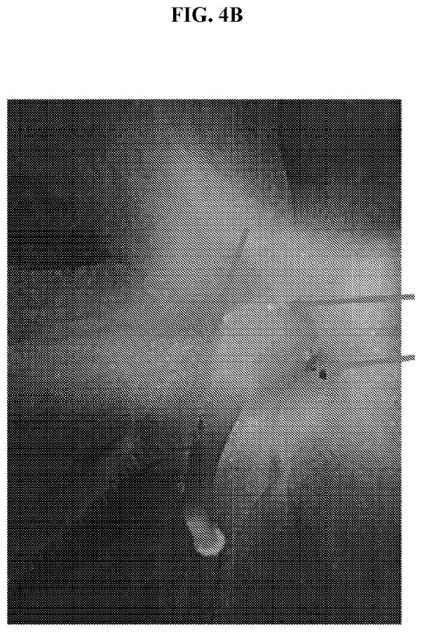

Therefore, the inventors developed a filantherless flower type, which is resistant to the troubling pest as well as pathogens (FIGS. 3, 4A, and 4B). Also provided is a method of producing a new African violet variety that produce the filantherless trait by crossing a first plant selection carrying the presence of some filantherless flowers trait with a second plant selection that does not carry the filantherless trait but exhibits a second trait of interest. The method also comprises selecting a progeny that exhibits filantherless trait and the second trait of interest.

In one embodiment, the filantherless flowers of this invention, when matured, are in absence of any filament or anthers visible on any of the flowers. The production of the filantherless trait includes selections for a flower that does not have any signs of a developed stamen, or a developed filament and anther in the first flash of flowers. In some embodiments, the flower remains filantherless at the first flash. In another embodiment, the second and subsequent generations of flowers start to show the developed stamen that features a developed filament and anthers under various growing conditions.

The plants of this invention can mature for sale without the troublesome anthers, which allows the growers to use less insecticides or fungicides to get the plant products ready for the market. They do not show any signs of developed stamen, filament and anther in the first flash of flowers. In one embodiment, the second and subsequent generations of flower can start to show the development of the stamen featuring a developed filament and anthers under various growing conditions.

Therefore, in one aspect, the disclosure provides a Saintpaulia plant comprising at least one flower that is absent of a stamen in the flower. In one embodiment, the flower lacks a filament and an anther during the first flash of the flower. In another embodiment, the filament and the anther develop during the second flash of the flower. In one embodiment, the plant is produced from seeds having ATCC deposit Accession No. PTA ______. In another embodiment, the flower is a mature flower. In another embodiment, the Saintpaulia plant develops a fully developed stamen with a filament and anther at the second or later flash of flowers.

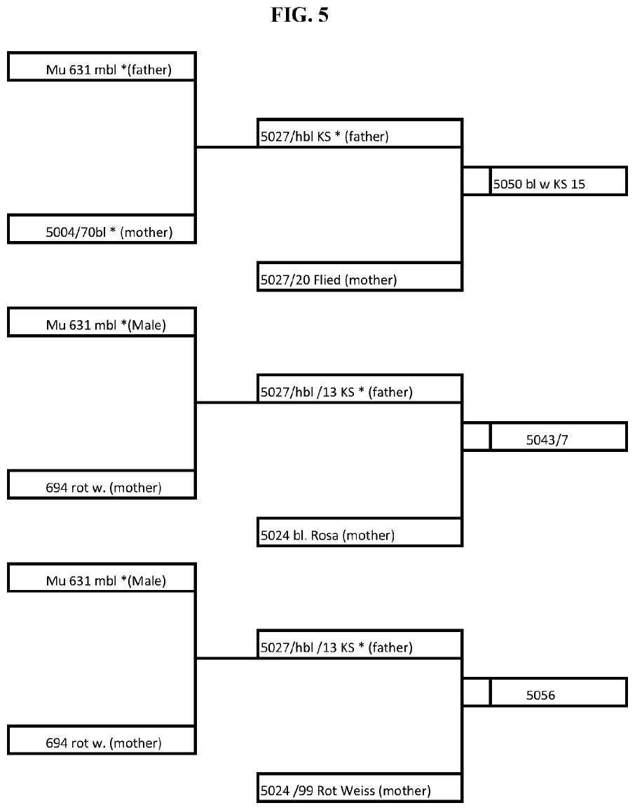

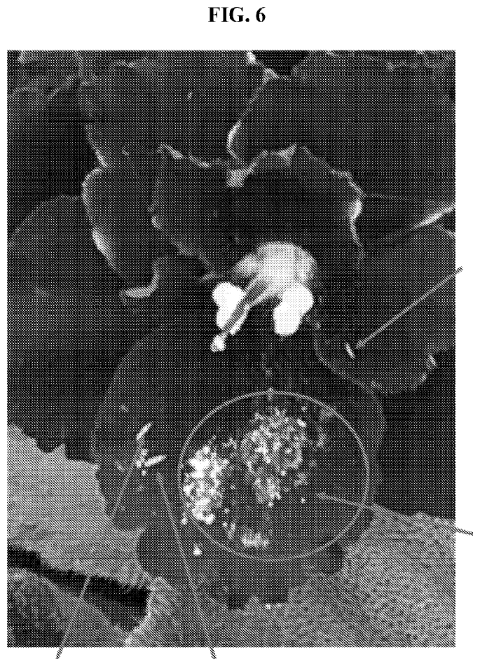

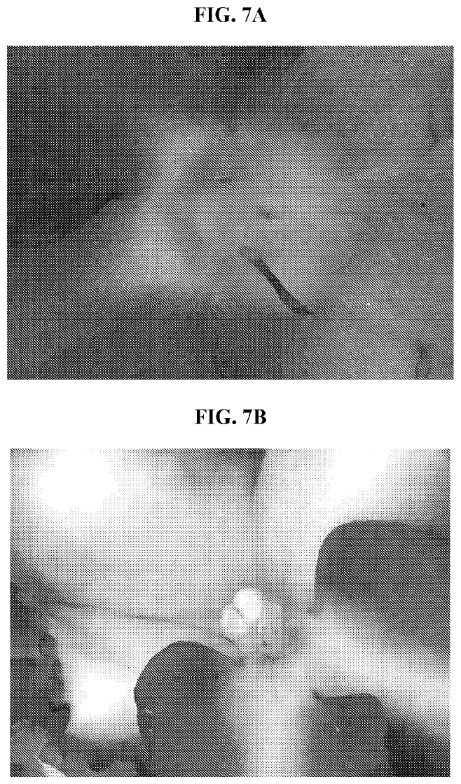

In another embodiment, the Saintpaulia plant is selected from a group consisting of a ‘5050 bl w KS 15’ cultivar, a ‘5043/7’ cultivar, and a ‘5056’ cultivar (FIG. 5). The presence of anthers with the troubling Western Flower Thrip (Frankliniella occidentalis) or the presence of the pathogen Botrytis is more troubling to keep the plants sellable (FIGS. 6, 7A, and 7B). Thus, the lack of stamen makes the African violet less susceptible to pathogen infection. In another embodiment, the Saintpaulia plant is less likely to be infected with a pathogen than a control. In one embodiment, the pathogen is botrytis and/or Frankliniella occidentalis.

The filantherless trait is firmly fixed in the breeding stock described herein and is retained through successive generations of asexual or sexual reproduction. The present invention also relates to introducing the filantherless trait into diverse Saintpaulia (African violet) cultivars to produce plants exhibiting the filantherless trait in combination with many different desirable characteristics. Non-limiting desirable characteristics include various petal colors, leaf shapes, leaf color, growth habit and others.

The project that resulted in production of the filantherless trait began in August 2010 at the research facility located in Isselburg, Germany. Among the interesting mutant phenotypes observed among the plants grown from the seeds was a single plant with the filantherless trait. This Saintpaulia plant of this invention that exhibited the filantherless trait has had at least one flower without the development of the stamen or lacks the filament and the anther. Preferably, the African violet plant exhibiting the filantherless trait has more than one flower without the development of the stamen. More preferably, an African violet plant exhibiting the filantherless trait has a mature flower head with no filament or anther development at all. In another embodiment, the filantherless African violet has a different desirable characteristic.

The Saintpaulia plant is produced by a breeding method for transmitting to new cultivars of African violets the trait or characteristic of the filantherless flower with the propagative material needed to accomplish this. Thus, in another aspect, the invention provides a method of producing a Saintpaulia plant that comprises at least one mature flower that is absent of a stamen in the flower, the method comprises crossing a first Saintpaulia plant that has a partial filament and/or an antherless characteristic with a second Saintpaulia plant having a second desirable trait. In one embodiment, the second Saintpaulia plant has a fully developed stamen. In another embodiment, the second Saintpaulia plant has a filament and an anther. In one embodiment, the second desirable trait is selected from the group consisting of flower color, leaf color, disease resistance, leaf size and growth habit.

The method, in one embodiment, further comprises producing a seed from the Saintpaulia plant that comprises at least one mature flower that is absent of a stamen in the flower, wherein the seed is capable of growing to a filantherless Saintpaulia plant. In another embodiment, the method further comprises selecting a progeny from the crossing, wherein the progeny produces more filantherless flowers than its parents.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### WORKING EXAMPLES

### Example 1 Production of a Filantherless African Violet Variety—‘5050 bl w KS 15’

The filantherless variety ‘5050 bl w KS 15’ was produced from the cross of ‘5027/hbl KS*’ as father and ‘5027/20 Flied’ as mother. Asexual reproduction of the new cultivar by leaf cuttings, as performed by the inventor in Nashville, Tenn., has demonstrated that the combination of characteristics as herein disclosed for the new cultivar were firmly fixed and retained through successive generations of asexual reproduction. Cuttings for asexual propagation can be taken at any time of the year, and no special hormones or soil mixtures are used.

Although the phenotype may vary significantly with variations in environment such as temperature, light intensity and day length, it has been determined that the filantherless trait is highly stable even with less than optimum growing conditions for the first flash of flowers. The following observations, measurements and values describe the new cultivar as grown in Nashville, Tenn., under greenhouse conditions which closely resemble those generally used in commercial practice.

The produced ‘5050 bl w KS 15’ cultivars had strong, upright flower stems curving slightly toward the center, small medium-blue flowers with a white center and a filantherless trait. The cultivar exhibited long-lasting, non-dropping flowers. The plant was saleable for 8 to 10 weeks after potting. The cultivar also exhibits vigorous and compact growth habit. Both ovum style and stigma were pushed through the center of the flower. No stamen, including a filament or an anther, was present on the cultivars. The leaves on the cultivar were medium green and oval to heart-shaped. After maturity, the flowers dried off and remained on the peduncle without being infected by Botrytis.

In Table 1, color references are made to the Royal Horticultural Society Colour Chart (RHS), except where general colors of ordinary significance are referred to. Color values were taken under natural sunlight conditions at approximately 12 p.m. in Nashville, Tenn.

TABLE 1
Description of the ‘5050 bl w KS 15’ cultivar

| | |
|---|---|
| Botanical | Saintpaulia ionantha |
| Classification | |
| c.v. 5050 bl | Male parent: ‘5027/hbl KS*’ |
| w KS | Female parent: ‘5027/20 Flied’ |
| Parentage | |
| Propagation | The new cultivar held its distinguishing characteristics |
| | through successive propagations by leaf cuttings. |
| Plant: | From 9-11 cm tall when grown in pots and |
| | 20-25 cm diameter when fully grown. |
| | Leaves: |
| | General form: Oval to Round-shaped |
| | Diameter: 60-70 mm wide and 75-85 mm long |
| | Texture: Leathery |
| | Aspect: Hairy, slightly serrated, slightly shiny |
| | Veins: Upper side: visible; underside: well pronounced, |
| | light green to purplish red, shiny |
| | Color (upper side): Yellow-Green RHS 147 A − 147 B |
| | Color (underside): Greyed-Green RHS 194 B − 194 C |
| | Petiole: Strong, green with purplish brown touch, hairy |
| | Flowers: |
| | Buds: Bell-shaped, light blue, 5-7 mm just before opening |
| | Sepals: Five |
| | Color: Green with purplish brown touch |
| | Calyx: |
| | Shape: Funnel-shaped |
| | Aspect: Spear-shaped, hairy Peduncle |
| | Character: Strong upright, hairy |
| | Color: Purplish brown |
| | Individual flowers: |
| | Size: 32-36 mm |
| | Shape: Single, violet-shaped |
| | Color (upper side): Between Violet-Blue RHS 89 A and |
| | RHS 89 B with center being white. Under cooler |
| | temperatures and more intensive lighting, the purple area |
| | tends to intensify with the white area of the center |
| | of the flower tending to recede. |
| | Color (underside): Violet RHS 85 A to RHS 85 B |
| | Borne: Each flower stem carried 10-13 and more flowers on |
| | strong, upright peduncles that were free standing above |
| | Flowering habit: |
| | Flowers 8-10 weeks after potting |
| | Reproductive Organs: No Stamens development with lack of |
| | the two anthers as well as the supporting filament |
| | A Styles: 6-7 mm long, light blue, base of ovary light green |
| | and hairy |
| | Roots: Normally developed, white when young, turning |
| | slightly brownish when older |
| | Disease Resistance: ‘5050 bl w KS 15’ has shown very good |
| | resistance to all major violet diseases. The lack of the anthers |
| | had the benefit that this cultivar was not desirable for the |
| | Western Flower Thrip or Frankliniella occidentalis The |
| | cultivar was also not susceptible to developing botlytis |
| | on the anther. |

The ‘5050 bl w KS 15’ was an attractive cultivar due to its abundance of blue flowers with a white center contrasting nicely. A tight flower bouquet was free-standing and above the leaves develops after 8-10 weeks. Each of the 10-12, or more, strong flower stems carried 10-13 or more flowers. Due to the filantherless characteristic, the flowers are resistant to various diseases or pathogens.

### Example 2 Production of the ‘5043/7’ Cultivar

The ‘5043/7’ cultivar is produced from the cross of selection ‘5027/hbl/13 KS*’ with ‘5024 bl. Rosa’. Asexual reproduction of the new cultivar by leaf cuttings, as performed by the inventor in Nashville, Tenn., demonstrated that the combination of characteristics as herein disclosed for the new cultivar were firmly fixed and retained through successive generations of asexual reproduction. Cuttings for asexual propagation can be taken at any time of the year, and no special hormones or soil mixtures are used.

The phenotype of ‘5043/7’ cultivar may vary significantly with variations in environment such as temperature, light intensity and day length; however, it has been determined that the filantherless characteristic is highly stable even with less than optimum growing conditions for the first flash of flowers.

The ‘5043/7’ cultivar had strong, upright flower stems curving slightly toward the center, small to medium size single blue flower and a filantherless trait. The cultivar exhibits long-lasting, non-dropping flowers. The plant is saleable for 8 to 10 weeks after potting. The cultivar exhibits vigorous and compact growth habit. Both the ovum style and stigma was pushed through the center of the flower. No stamen is present on the cultivar. The leaves are medium green with oval to heart shapes. After maturity, the flowers dry off and remain on the peduncle without being infected by Botrytis.

In Table 2, color references of the cultivar are made to the Royal Horticultural Society Colour Chart (RHS), except where general colors of ordinary significance are referred to. Color values are taken under natural sunlight conditions at approximately 12 p.m. in Nashville, Tenn.

TABLE 2
Description of the ‘5043/7’ cultivar

| | |
|---|---|
| Botanical | Saintpaulia ionantha |
| Classi- | |
| fication | |
| c.v. 5043/7 | Male parent: ‘5027/hbl KS *’ |
| Parentage | Female parent: ‘5024 bl. Rosa’ |
| Propa- | The new cultivar held its distinguishing characteristics |
| gation | through successive propagations by leaf cuttings. |
| Plant: | Leaves |
| | From 9-11 cm tall when grown in pots and 20-25 cm |
| | diameter when fully grown. |
| | General form: Oval to heart-shaped |
| | Diameter: 60-70 mm wide and 75-85 mm long |
| | Texture: Leathery |
| | Aspect: Hairy, slightly serrated, slightly shiny |
| | Veins: |
| | Upper side: visible; |
| | Underside: well pronounced, light green to purplish red, shiny |
| | Color (upper side): Yellow-Green RHS 147 A |
| | Color (underside): Greyed-Green RHS 195 B with touches of |
| | Greyed-Purple RHS 186 C to RHS 186D |
| | Petiole: Strong, green with purplish brown touch, hairy |
| | Flowers: |
| | Buds: Bell-shaped, light blue, 5-7 mm just before opening |
| | Sepals: Five |
| | Color: Green with purplish brown touch |
| | Calyx: |
| | Shape: Funnel-shaped |
| | Aspect: Spear-shaped, hairy Peduncle |
| | Character: Strong upright, hairy |
| | Color: Purplish brown |
| | Individual flowers: |
| | Size: 25-32 mm |
| | Shape: Single, violet-shaped |
| | Color (upper side): Between Violet-Blue RHS 89 A and |
| | RHS 89 B with center being white. Under cooler temperatures |
| | and more intensive lighting, the purple area tends to intensify |
| | with the white area of the center of the flower tending to recede. |
| | Color (underside): Between Violet-Blue RHS 89 A |
| | Color (underside): Violet RHS 88 A |
| | Borne: Each flower stem carried 10-13 and more flowers on |
| | strong, upright peduncles that are free standing above |
| | Flowering habit: |
| | Flowers 8-10 weeks after potting |
| | Reproductive Organs. No Stamens development with lack of |
| | the two anthers as well as the supporting filament |
| | A Styles: 6-7 mm long, light blue, base of ovary light green |
| | and hairy. |
| | Roots: Normally developed, white when young, turning slightly |
| | brownish when older |
| | Disease Resistance: ‘5043/7’ has shown very good resistance to |
| | all major violet diseases. The lack of the Anthers has the benefit |
| | that this cultivar is not desirable for the Western Flower Thrip |
| | or Frankliniella occidentalis. The Cultivar is also not |
| | susceptible to developing botrytis on the anther. |

The ‘5043/7’ cultivar is an attractive cultivar due to its abundance of blue flowers. A tight flower bouquet is free-standing and above the leaves develops after 8-10 weeks. Each of the 10-12 or more of strong flower stems carries 10-13 or more flowers. Due to its filantherless characteristic, the flowers are resistant to various diseases and/or pathogens.

### Example 3 Production of a Filantherless African Violet Variety—‘5056’

The filantherless variety ‘5056’ is produced from the cross of ‘5027/hbl KS*’ as father and ‘5024/99 Rot Weiss’ as mother. Asexual reproduction of the new cultivar by leaf cuttings, as performed by the inventor in Nashville, Tenn., has demonstrated that the combination of characteristics as herein disclosed for the new cultivar were firmly fixed and retained through successive generations of asexual reproduction. Cuttings for asexual propagation can be taken at any time of the year, and no special hormones or soil mixtures are used.

Although the phenotype may vary significantly with variations in environment such as temperature, light intensity and day length, it has been determined that the filantherless trait is highly stable even with less than optimum growing conditions for the first flash of flowers.

The produced ‘5056’ cultivars have strong, upright flower stems curving slightly toward the center, small or medium-reddish flowers with a white center and a filantherless trait. The cultivar exhibits long-lasting, non-dropping flowers. The plant is saleable for 8 to 10 weeks after potting. The cultivar also exhibits vigorous and compact growth habit. Both ovum style and stigma are pushed through the center of the flower. No stamen, including a filament or an anther, is present on the cultivars. The leaves on the cultivar are medium green and oval to heart-shaped. After maturity, the flowers dry off and remain on the peduncle without being infected by Botrytis.

In Table 3, color references are made to the Royal Horticultural Society Colour Chart (RHS), except where general colors of ordinary significance are referred to. Color values are taken under natural sunlight conditions at approximately 12 p.m. in Nashville, Tenn.

Description of the ‘5056’ cultivar

| | |
|---|---|
| Botanical | Saintpaulia ionantha |
| Classi- | |
| fication | |
| c.v. 5056 | Male parent: ‘5027/hbl KS *’ |
| Parentage | Female parent: ‘5024 /99 Rot Weiss’ |
| Propa- | The new cultivar held its distinguishing characteristics |
| gation | through successive propagations by leaf cuttings. |
| Plant: | From 9-11 cm tall when grown in pots and 20-25 cm |
| | diameter when fully grown. |
| | Leaves |
| | General form: Oval to heart-shaped |
| | Diameter: 60-70 mm wide and 75-85 mm long |
| | Texture: Leathery |
| | Aspect: Hairy, slightly serrated, slightly shiny |
| | Veins: Upper side: visible; underside: well pronounced, |
| | light green to purplish red, shiny |
| | Color (upper side): Yellow-Green RHS 147 A to RHS 147 B |
| | Color |
| | Color (underside): Greyed-Green RHS 195 B to RHS 195 C |
| | Petiole: Strong, green with purplish brown touch, hairy |
| | Flowers: |
| | Buds: Bell-shaped, light blue, 5-7 mm just before opening |
| | Sepals: Five |
| | Color: Green with reddish burgundy touch |
| | Calyx: |
| | Shape: Funnel-shaped |
| | Aspect: Spear-shaped, hairy Peduncle |
| | Character: Strong upright, hairy |
| | Color: reddish burgundy |
| | Individual flowers: |
| | Size: 20-30 mm |
| | Shape: Single, violet-shaped |
| | Color (upper side): Between Purple-Violet RHS 81 A and |
| | RHS 80 A. Under cooler temperatures and more intensive |
| | lighting, the light red tends to intensify. |
| | Color (underside): Purple-Violet RHS 81 B and RHS 81 D |
| | Borne: Each flower stem carried 10-13 and more flowers on |
| | strong, upright peduncles that were free standing above |
| | Flowering habit: |
| | Flowers 8-10 weeks after potting |
| | Reproductive Organs. No Stamens development with lack of |
| | the two anthers as well as the supporting filament |
| | A Styles: 6-7 mm long, reddish pink, base of ovary light green |
| | and hairy. |
| | Roots: Normally developed, white when young, turning |
| | slightly brownish when older. |
| | Disease Resistance: The ‘5043/7’ has shown very good |
| | resistance to all major violet diseases. The lack of the anthers |
| | had the benefit that this cultivar was not desirable for the |
| | Western Flower Thrip or Frankliniella occidentalis. The |
| | cultivar was also not susceptible to developing botrytis |
| | on the anther. |

The ‘5056’ is an attractive cultivar due to its abundance of reddish flowers with a white center contrasting nicely. A tight flower bouquet is free-standing and the leaves develops after 8-10 weeks. Each of the 10-12 or more of the strong flower stems carries 10-13 or more flowers. Due to the filantherless characteristic, the flowers are resistant to various diseases or pathogens.

In this manner described above, the filantherless trait has been successfully fixed in the breeding materials so that the filantherless trait can be easily and predictably bred into diverse African violet genetic backgrounds. The filantherless trait has been successfully combined with a large number of desirable African violet characteristics including different flower colors, leaf colors and growth habit. The different cross-breeding methods have transmitted the filantherless characteristic approximately 50% of the time in crosses including one parent exhibiting filantherless trait and approximately 80% of the time in crosses when both parents exhibit the filantherless trait.

Surprisingly, the filantherless trait regressed with the age of the plants. The first flash of flowers was typically 99.9% filantherless with only a very small number of individual flowers showing slight development of the stamen. Subsequent flower cycles after the first cycle have a higher percentage of flowers developing the stamen with a fully developed filament and anther. This in particular is valuable since the plant reverted partially back to the traditional African violet flower featuring both sexes.

### EQUIVALENTS

It is to be understood that while the disclosure has been described in conjunction with the above embodiments, the foregoing description and examples are intended to illustrate and not limit the scope of the disclosure. Other aspects, advantages and modifications within the scope of the disclosure will be apparent to those skilled in the art to which the disclosure pertains.

Unless otherwise defined, all technical and scientific terms used herein have the same meaning as commonly understood by one of ordinary skill in the art to which this disclosure belongs.

The embodiments illustratively described herein may suitably be practiced in the absence of any element or elements, limitation or limitations not specifically disclosed herein. Thus, for example, the terms “comprising,” “including,” “containing,” etc., shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure.

Thus, it should be understood that, although the present disclosure has been specifically disclosed by specific embodiments and optional features, modification, improvement and variation of the embodiments herein disclosed may be resorted to by those skilled in the art, and that such modifications, improvements and variations are considered to be within the scope of this disclosure. The materials, methods and examples provided here are representative of particular embodiments, are exemplary and are not intended as limitations on the scope of the disclosure.

The scope of the disclosure has been described broadly and generically herein. Each of the narrower species and subgeneric groupings falling within the generic disclosure also form part of the disclosure. This includes the generic description with a proviso or negative limitation removing any subject matter from the genus, regardless of whether or not the excised material is specifically recited herein.

In addition, where features or aspects of the disclosure are described in terms of Markush groups, those skilled in the art will recognize that embodiments of the disclosure may also thereby be described in terms of any individual member or subgroup of members of the Markush group.

All publications, patent applications, patents and other references mentioned herein are expressly incorporated by reference in their entirety, to the same extent as if each were incorporated by reference individually. In case of conflict, the present specification, including definitions, will control.

1. A Saintpaulia plant comprising at least one flower that is absent of a stamen in the flower.

2. The Saintpaulia plant of claim 1, wherein the flower lacks a filament and an anther during the first flash of the flower.

3. The Saintpaulia plant of claim 1, wherein the filament and the anther develop during the second flash of the flower.

4. The Saintpaulia plant of claim 1, wherein the plant is produced from a seed having ATCC deposit Accession No. PTA ______.

5. The Saintpaulia plant of claim 1, where the flower is a mature flower.

6. The Saintpaulia plant of claim 1, wherein Saintpaulia plant develops a fully developed stamen with a filament and anther at the second or later flash of flowers.

7. The Saintpaulia plant of claim 1, wherein the Saintpaulia plant is less likely to be infected with a pathogen than a control.

8. The Saintpaulia plant of claim 7, wherein the pathogen is botrytis and/or Frankiniella occidentalis.

9. The Saintpaulia plant of claim 1, wherein the Saintpaulia plant is selected from a group consisting of a “5050 bl w KS 15” cultivar, a “5043/7” cultivar and a “5056” cultivar.

10. A method of producing a Saintpaulia plant that comprises at least one mature flower that is absent of a stamen in the flower, the method comprises crossing a first Saintpaulia plant that has partial filament and antherless characteristics with a second Saintpaulia plant having a second desirable trait.

11. The method of claim 10, wherein the second Saintpaulia plant has a fully developed stamen.

12. The method of claim 10, wherein the second Saintpaulia plant has a filament and an anther.

13. The method of claim 10, wherein the second desirable trait is selected from the group consisting of flower color, leaf color, disease resistance, leaf size and growth habit.

14. The method of claim 10, further comprises producing a seed from the Saintpaulia plant that comprises at least one mature flower that is absent of a stamen in the flower, wherein the seed is capable of growing to a filantherless Saintpaulia plant.

15. The method of claim 10, further comprises selecting a progeny from the crossing, wherein the progeny produces more filantherless flowers than its parents.
